(19) United States
(12) Patent Application Publication
(10) Pub. No.: US 2013/0289414 A1
(43) Pub. Date: Oct. 31, 2013

(54) COMBINED ABSORPTION-REFLECTION BASED INSTRUMENT AND TECHNIQUE TO MEASURE ANTIOXIDANTS (INCLUDING CAROTENOIDS) IN HUMAN TISSUE

(71) Applicants: Mahmoudreza Adibnazari, Salt Lake City, UT (US); Omid Adibnazari, Salt Lake City, UT (US)

(72) Inventors: Mahmoudreza Adibnazari, Salt Lake City, UT (US); Omid Adibnazari, Salt Lake City, UT (US)

(21) Appl. No.: 13/791,237

(22) Filed: Mar. 8, 2013

Related U.S. Application Data
(60) Provisional application No. 61/609,013, filed on Mar. 9, 2012.

(51) Int. Cl. A61B 5/00

(52) U.S. Cl. CPC A61B 5/443
USPC 600/476

(57) ABSTRACT

A combined absorption-reflection based instrument and methods are introduced here to measure antioxidant carotenoids and similar compounds such as: beta-carotene, lycopene and lutein, along with others in living human tissue (e.g. skin). The device and methods provide a non-invasive, rapid, accurate, repeatable, safe and reliable method for measuring antioxidant levels in human tissue, providing information that can be used for many diagnostic and/or health purposes.

### CROSS-REFERENCE TO RELATED APPLICATIONS

This application claims priority to U.S. Provisional Application Ser. No. 61/609,013, filed Mar. 9, 2012, and entitled “A Combined Absorption-Reflection Based Instrument and Technique to Measure Antioxidants (including Carotenoids) in Human Tissue,” the contents of which are incorporated herein by reference in their entirety.

### BACKGROUND

1. The Field of the Invention

The present invention relates to combining reflection and absorption spectroscopy to measure characteristics of human tissue by measuring antioxidant and related compound levels. The apparatus and methods disclosed herein are highly accurate, fast, non-invasive, reliable, and provides the subject with a measure of antioxidant levels, which are highly correlated with the subject's health.

2. The Relevant Technology

The present invention relates to measuring levels of antioxidant and related compounds in human tissue.

### BRIEF SUMMARY

A combined absorption-reflection based instrument and methods are introduced here to measure antioxidant carotenoids and similar compounds such as: beta-carotene, lycopene and lutein, along with others in living human tissue (e.g. skin). The device and methods provide a non-invasive, rapid, accurate, repeatable, safe and reliable method for measuring antioxidant levels in human tissue, providing information that can be used for many diagnostic and/or health purposes.

Absorption spectroscopy is a popular method: it compares the level of the input light entering a solution and the output light based on Beer-Lambert's law, which measures the concentration level of any compound in such solution (the solution must be not highly concentrated).

Another popular tool is the reflection spectroscopy technique, which compares the level of the incident light shined on a sample with the diffused, back scattered light to measure the concentration level of the desired compound. Reflection spectroscopy is also based on Beer-Lambert's law.

Furthermore, Beer-Lambert's law is not valid in highly scattered and/or absorbance media, like human tissue. Many attempts have been made to modify this law to approximate the needed concentration level in a media.

In the current approach we introduce a simple method based on both effects—absorption and reflection—that is not limited to the necessary conditions in Beer-Lambert's law. Using this method, heretofore referred to as the “combined method,” it is possible to successfully measure the relative concentration of antioxidants in the subject's tissue and compare the antioxidant levels with other subjects.

This method does not compare the output light (after it is absorbed or diffused back scattered) with the input light; rather, it compares the output light with the source light spectrum.

Also worth noting, this system uses a wide band illumination light source projected on the surface of tissue, such as human palm or thumb skin, to calculate the change of the light source spectrum through absorption of all major absorptive materials in tissue such as melanin, hemoglobin and carotenoids, which ultimately provides us with the level of carotenoids in tissue.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 is a schematic diagram of the combined Abs-Ref instrument according to the present invention.

FIG. 2 illustrates the compact Abs-Ref instrument measuring the tissue in details.

FIG. 3 illustrates the compact Abs-Ref instrument measuring the human palm.

FIG. 4 illustrates the compact Abs-Ref instrument measuring the human arm.

FIG. 5 illustrates the compact Abs-Ref instrument measuring the human palm.

FIG. 6 demonstrates the concept of absorption (upper section) and Abs-Ref methods.

FIG. 7 shows the human skin tissue layers (upper section) and the Abs-Ref method applying on the human skin tissue.

FIG. 8 plots carotenoid beta-carotene absorption spectra in UV-VIS range as the major antioxidant in human body.

FIG. 9 illustrates carotenoid beta-carotene molecular structure.

FIG. 10 plots deoxyhemoglobin absorption spectra in UV-VIS range.

FIG. 11 plots oxyhemoglobin absorption spectra in UV-VIS range.

FIG. 12 plots melanin pigment absorption spectra in UV-VIS range.

FIG. 13 plots deoxyhemoglobin, oxyhemoglobin, antioxidant beta-carotene and melanin absorption spectra in UV-VIS range.

FIG. 14 plots melanin the wavelength region of interest to measure antioxidant beta-carotene level in the present of the other pigments in human skin tissue in UV-VIS range.

FIG. 15 demonstrates the human skin tissue spectra deduced from Abs-Ref method, showing three major pigments, antioxidant beta-carotene, deoxyhemoglobin and oxyhemoglobin, and the wavelength region of interest to measure antioxidant beta-carotene concentration.

FIG. 16 shows the algorithm of the Abs-Ref method measuring antioxidant beta-carotene concentration in human tissue.

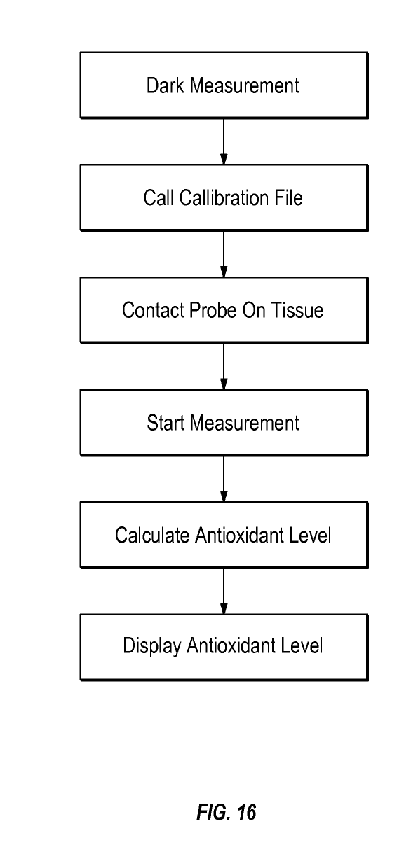

### DETAILED DESCRIPTION

Antioxidant Benefits for Human Body.

Numerous studies have been attempting to assess the role of carotenoids in human health. Many of these studies involve subjects eating a diet rich in antioxidant carotenoids from fruits or vegetables or simply by pure compound dietary supplements consisting primarily of beta-carotene, lycopene and lutein. More than 600 carotenoids are currently isolated from natural sources. Humans and other mammals are incapable of synthesizing carotenoids, emphasizing the need to obtain them through proper diet.

It turns out that antioxidant carotenoids have two main abilities: light harvesting, which protects human skin against harmful UV radiation, and quenching the reactive oxygen species (ROS) around the human body, which helps prevent certain cancers and may lower the risk of brain and eye diseases (like macular degeneration).

Carotenoids are a highly colored group of plant pigments that are known to be potent antioxidants. There are more than 600 different carotenoids, including well known compounds such as beta-carotene, lycopene, lutein, and zeaxanthin. Numerous studies have shown the preventative properties of carotenoids against cancers (e.g. prostate cancer), vascular diseases and eye diseases (e.g. cataract and macular degeneration).

It should be noted that dietary intake of colorful fruits such as yellow squash, carrots, mangos, watermelon, pink grapefruit, guava, and apricots, as well as green and leafy vegetables, can supply noteworthy amounts of antioxidant carotenoids.

Major Pigments in Human Skin.

Melanin.

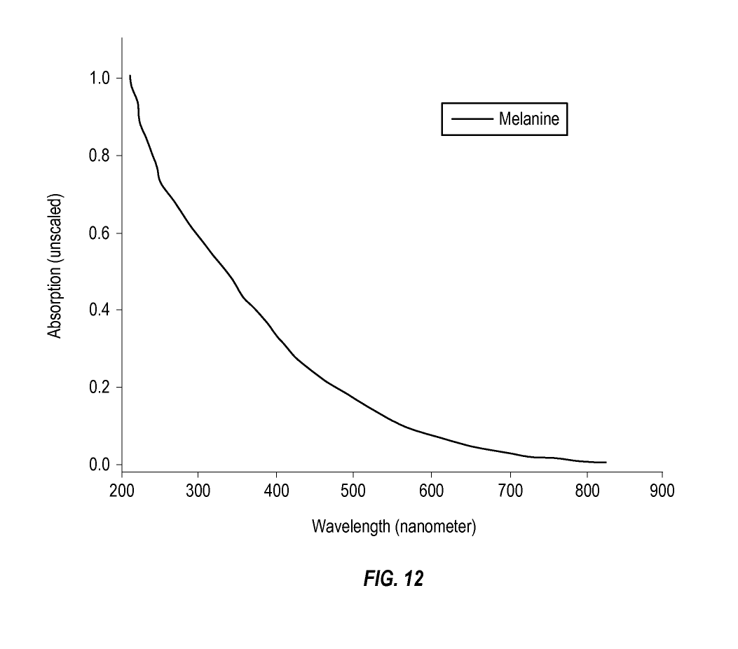

Chromophore melanin, which exists in the skin's epidermal layer, is responsible for protection against harmful UV radiation. Melanin is one of the major absorbers of light in some biological tissue, though its contribution is smaller than other components. The two types of melanin are eumelanin, which is black-brown and pheomelanin, which is red-yellow. The molar extinction coefficient spectra corresponding to both types are shown in FIG. 12.

Hemoglobin.

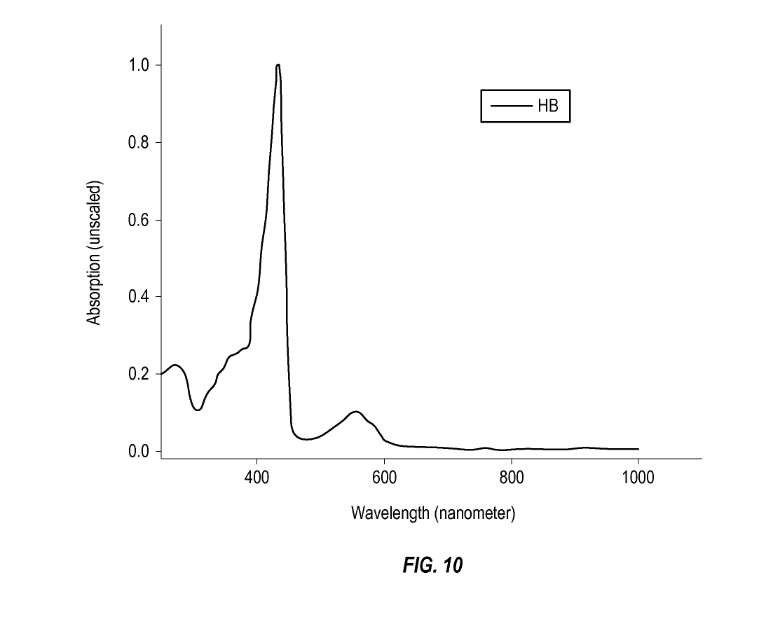

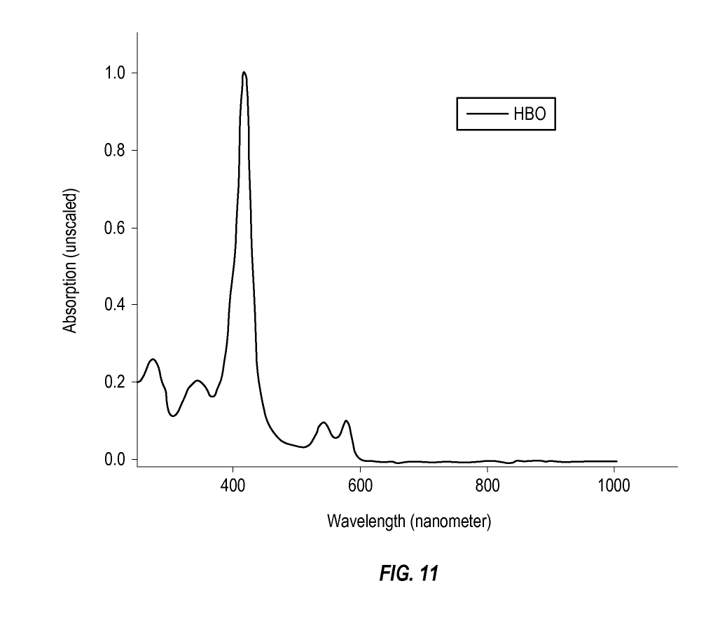

Blood consists of two different types of hemoglobin: oxyhemoglobin (HBO), which is bound to oxygen, and deoxyhemoglobin (HB), which is not. These two different types of hemoglobin exhibit different absorption characteristics, which are usually plotted as molar extinction coefficient or absorbance as functions of wavelength, as shown in FIGS. 10 and 11. The molar extinction coefficient of HB has its highest absorption peak around 435 nm and a second peak around 555 nm. Its spectrum then gradually decreases inversely, in proportion to the wavelength. On the other hand, HBO shows its highest absorption peak around 415 nm, and two secondary peaks around 540 nm and 575 nm. As light wavelengths pass 600 nm, HBO absorption decreases faster than HB absorption.

Beta-Carotene.

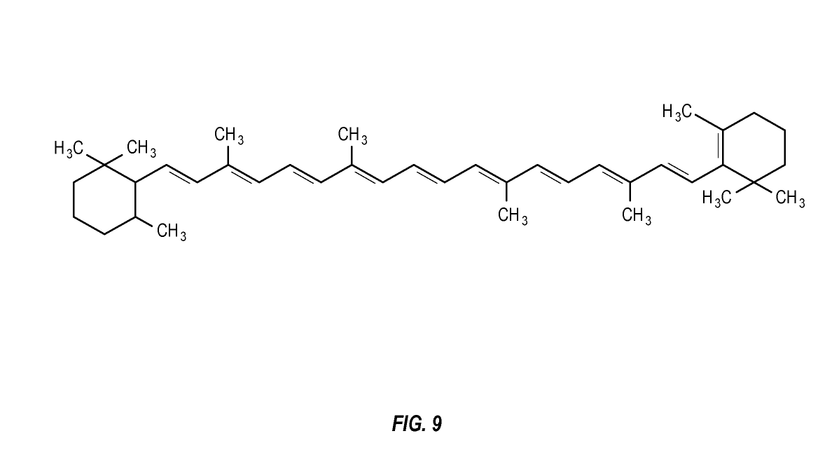

Beta-carotene is a well-known and abundant member of the carotenoid family. As illustrated in FIG. 9, beta-carotene is made up of eight isoprene units, which are cyclical at each end. These are joined end-to-end to give a conjugated long chain that is common to all carotenoids (beta-carotene has 40 carbons in this conjugated chain). This long conjugated chain is responsible for its strong red-orange color. Among this general class of carotenes, beta-carotene is distinguished by its beta-rings at each end of the molecule. Beta-carotene is a non-polar compound and has lipophilic properties. There are also other kinds of carotene, such as antioxidant alpha and gamma carotenes, which can be converted to active vitamin A. Commercial beta-carotene is most commonly produced synthetically or extracted from palm oil, algae or fungi. Beta-carotene, like other carotenoids, is involved in light harvesting to participate in the energy transfer process in photosynthesis. From the visible spectrum, carotenoids absorb light in the blue-green wavelength range (400-500 nm).

Human Skin.

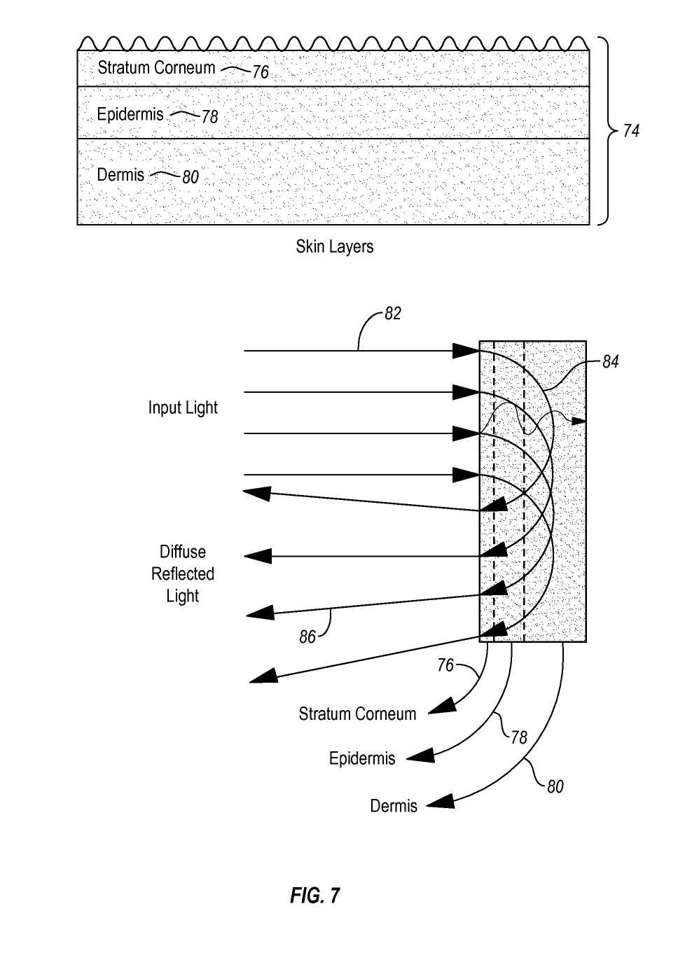

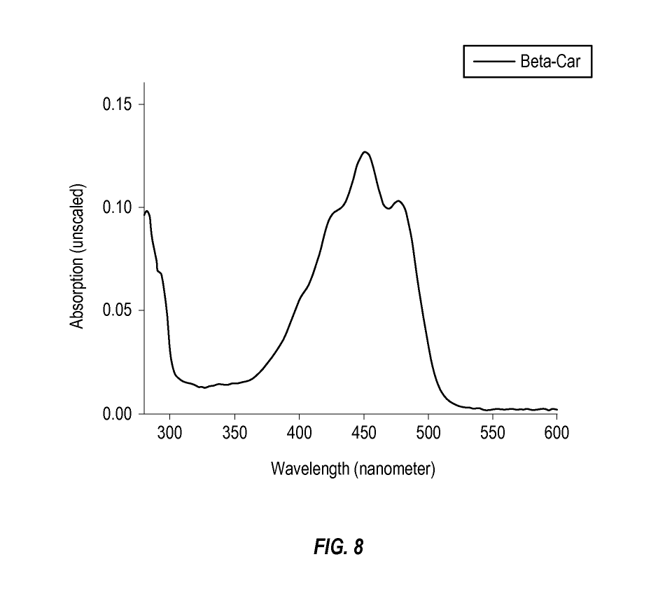

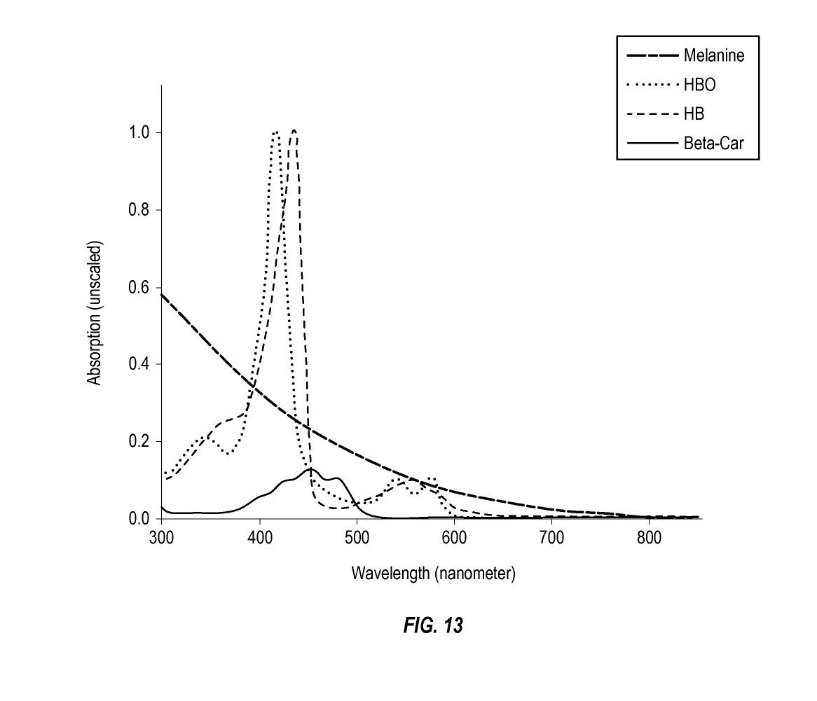

The skin, the largest organ of the body, serves three important functions: thermoregulation, sensation and protection. This organ consists of three main layers, the epidermis, the dermis, and the subcutaneous tissue, which is further subdivided into five layers. This invention focuses on the dermis, epidermis and the stratum corneum, the outer layer of the subcutaneous tissue (see FIG. 7). The stratum corneum is very thin, about 10-20 μm, and is composed of non-living corneocyte cells. The epidermis thickness is about 100 μm and the dermis is thicker than the epidermis, having approximately one-millimeter of thickness. There are also other deeper layers, which are not related to this invention. Most skin disorders result from ultraviolet light exposure. The main human skin pigments (hemoglobin, melanin, and carotenoids) are extremely ultraviolet absorbers, and they also absorb light from the blue-green wavelength range to some extent (see FIGS. 8, 10-15).

Absorption Spectroscopy.

One of the most popular, albeit archaic, methods for measuring a particular compound's concentration in a gas or low-concentration solution is absorption spectroscopy. In the context of our invention, absorption spectroscopy refers to spectroscopic techniques that measure the absorption of radiation (as a function of frequency or wavelength) and its interaction with a sample. The sample absorbs energy, (i.e., photons) from the radiating field, which may be between the UV-VIS-IR. The intensity of the absorption varies as a function of wavelength, and this variation is the absorption spectrum. In other words, absorption spectroscopy determines the presence of a particular substance in a sample as well as the substance's concentration.

From a modern physics perspective, the absorption spectrum is principally determined by the atomic and molecular structure of a specific sample, meaning radiation is absorbed at wavelengths that correspond with the energy difference between the two quantum physics states of the molecules in the sample.

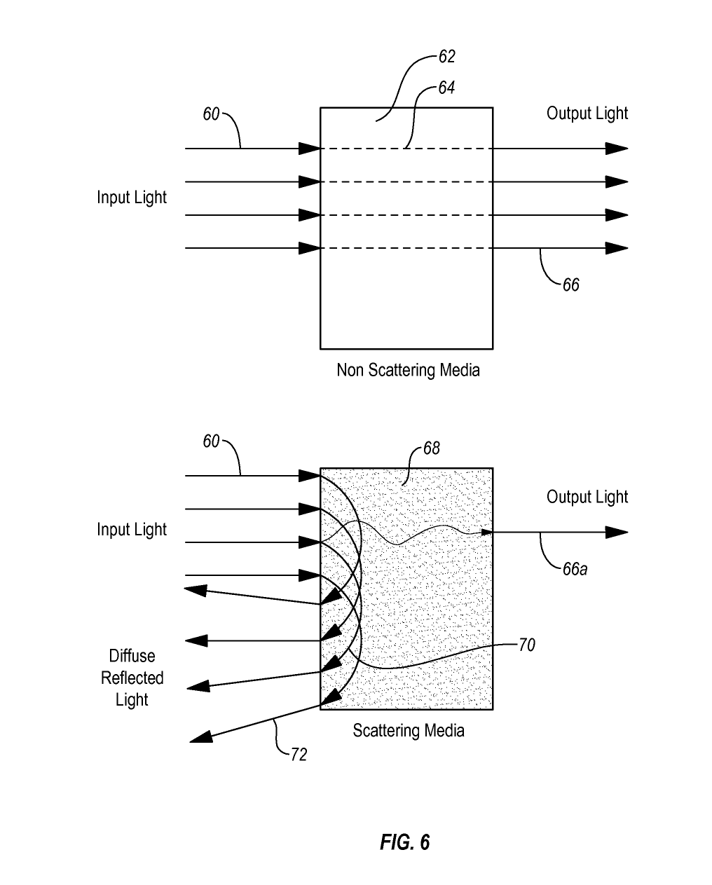

Absorption spectroscopy is graphically illustrated in FIG. 6. Usually when we want to measure absorption spectra, we project an incident beam of radiation (“Input Light”) 60 at a material 62 and compare the detected intensity of the radiation that passes through 64 it (“Output Light”) 66 with the incident beam 60, and then calculate the absorption level.

There are three main elements in absorption spectroscopy: the source, the material and the detector. These elements could vary drastically depending on the purpose of the measurement.

It should be noted that absorption and transmission spectra represent equivalent information and one can be calculated from the other through a mathematic transformation. A transmission spectrum will have its maximum intensities at wavelengths where the absorption is weakest because more light is transmitted through the sample; consequently, an absorption spectrum will have its maximum intensities at wavelengths where the absorption is strongest.

Beer-Lambert's Law.

The relation between the absorption of light and the properties of the material through which the light is traveling is referred to as Beer-Lambert's law. Beer-Lambert's law states that there is a logarithmic dependence between the transmission of light through a substance, the wavelength-dependent absorption coefficient, and the distance the light travels through the material. The absorption coefficient itself is a result of the extinction coefficient and concentration of the absorber in the material. There are different versions of this law for different phases of materials.

Generally Beer-Lambert's law could be written as:

A=Logarithm(Iᵢₙₚᵤₜ/Iₒᵤₜₚᵤₜ)=αl=∈lc

Where Iᵢₙₚᵤₜ and Iₒᵤₜₚᵤₜ are the incident and the transmitted light intensity, respectively, A is absorbance, α is wavelength-dependent absorption coefficient, l is path length, ∈ is extinction coefficient and c is concentration. Thus, if the logarithm ratio is known, the absorbance could be measured. Likewise, concentration can be determined if absorbance, extinction coefficient and path length are known. Usually absorbance becomes linear with the concentration in gases and liquids with low concentration. Unfortunately, this elegant linearity is spoiled as the concentration and/or scattering increases. There have been many attempts to modify this law to produce acceptable approximations for high concentrated absorbers within a given sample. The products of these attempts, however, have been controversial.

Reflection Spectroscopy.

Sometimes it is difficult to study samples using absorption spectroscopy, as the transmission's reliability can be obscure. It is because of the dependence of the measured intensity on both the scattering and absorption processes that causes this uncertainty (i.e. hitherto, there were no means to measure these properties individually). This leaves the alternative method of reflectance spectroscopy. Usually there is no need to prepare samples for this method. Reflectance measurements can be divided into the two basic categories of specular reflection and diffuse reflection. Diffuse reflectance spectroscopy has been a useful tool for in vivo studies of tissues in both diagnostic and therapeutic applications.

Diffuse reflection is the reflection of an incident light beam 60 (FIG. 6) from a surface 68 such that an incident light 60 is reflected at many angles 70 producing diffuse reflected light 72 rather than at just a single angle, which is the case in specular reflection. It should be noted that in diffuse reflection, it is not merely the surface that reflects the light; rather, most of the light is contributed through scattering beneath the surface (i.e., human tissue), which is graphically illustrated in FIG. 7. In FIG. 7, the example the human tissue is skin 74, which includes a stratum corneum layer 76, and epidermis layer 78, and a dermis layer 80. In this technique, incident light 82 is passed through and scattered 84 (i.e., absorbed and reflected) by the skin 74, which produces diffuse reflected light 86. The diffuse reflected light 86 contains information about certain chemicals (e.g., carotenoids) present in the skin 74 and at what concentration(s) they are present. The diffuse reflected light 86 is usually compared with a white standard (e.g., the incident light 82) in order to measure the concentration of the desired sample.

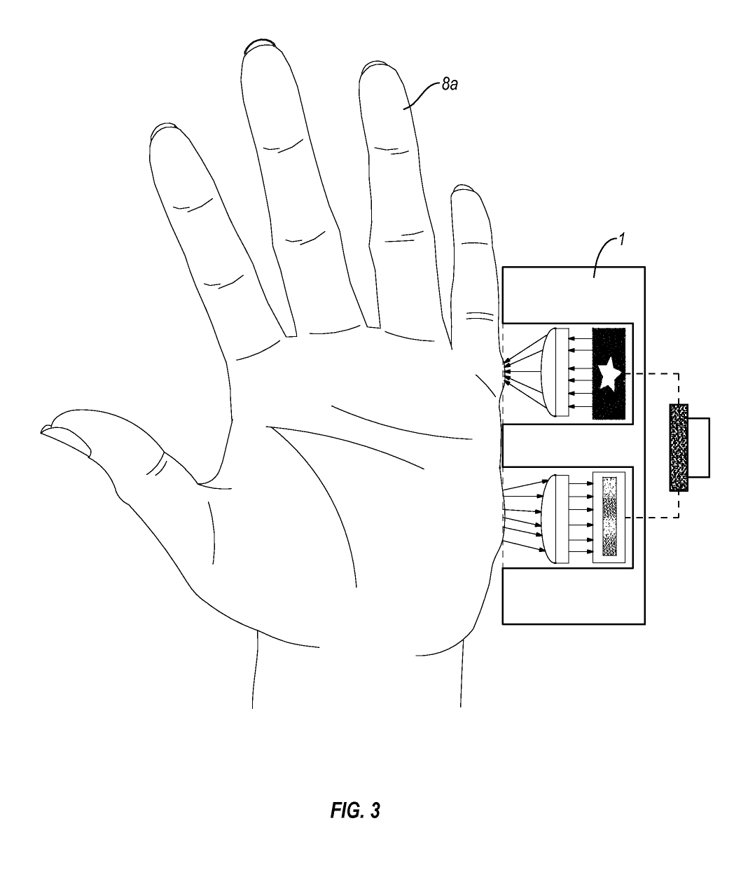

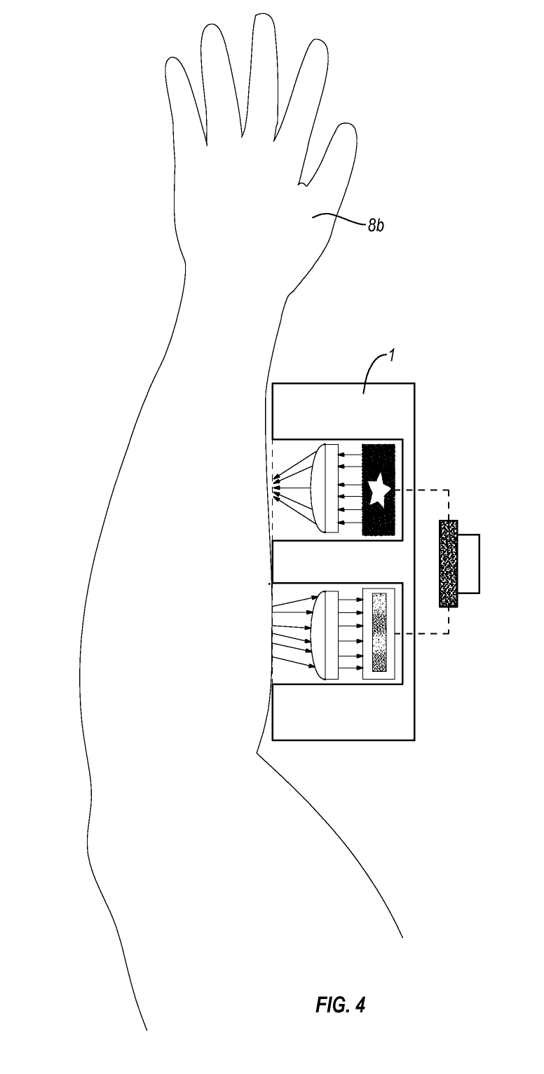

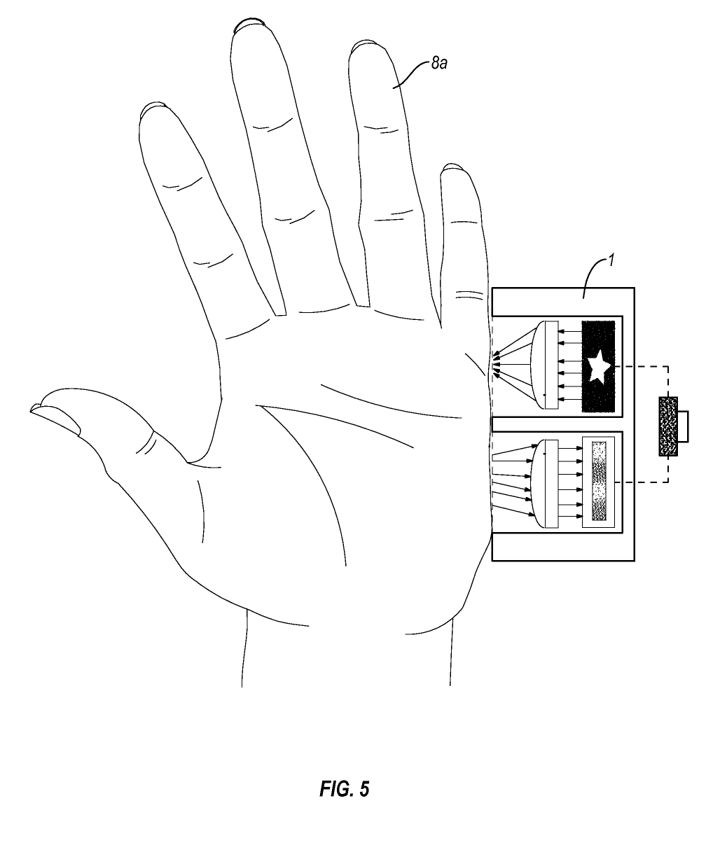

The present invention uses absorption and reflection spectroscopy to identify and quantify the presence of certain chemical compounds, namely antioxidant carotenoids and similar substances, in biological tissue, including human skin. The device directs light onto the tissue being studied. After the light travels a short distance inside the tissue, the instrument collects the diffused scattered light and compares it with the incident light spectrum. The diffused scattered light includes information like apparent absorption, which can be used to determine the concentrations of various chemical compounds present in a subject's tissue. The obtained apparent absorption can be expected to scale linearly with the concentration of any chemical compound of interest present in the sample volume of tissue. The use of a device (1) working in this way would be made effortless if its optical window could simply be pressed against human tissue sites such as the forearm (see 8b in FIG. 4) or palm of the hand (see 8a in FIGS. 3 and 5), and a rapid measurement subsequently taken. The apparatus allows continuous measure and display of the strength of apparent absorption. Using this device, the total time it would take to assess the concentration of a particular chemical compound in a subject's tissue would be very brief, amounting to only a few seconds.

In this method for noninvasive measurement of chemical compounds, such as antioxidant carotenoids, in biological tissue, a light source such as a tungsten-halogen lamp, a light emitting diode, or natural sunlight is used. These light sources feature sufficiently high intensity at the spectral locations in the wavelength range where absorption bands of the chemical compounds of interest occur, such as in the 400 to 520 nm spectral region for carotenoids. When the diffused scattered light from the tissue is compared with the incident light, the obtained apparent absorption or optical density of the noteworthy chemical compound is proportional to its concentration in the illuminated tissue volume. Thus, the apparent absorption of such chemical compounds in biological tissue can be used as an optical measure of concentration, and this information can be used to assess various aspects of the tissue's vitality. The concentration levels of any chemical compound can be compared with levels of standard biological tissue to assess the risk or presence of a malignancy disease.

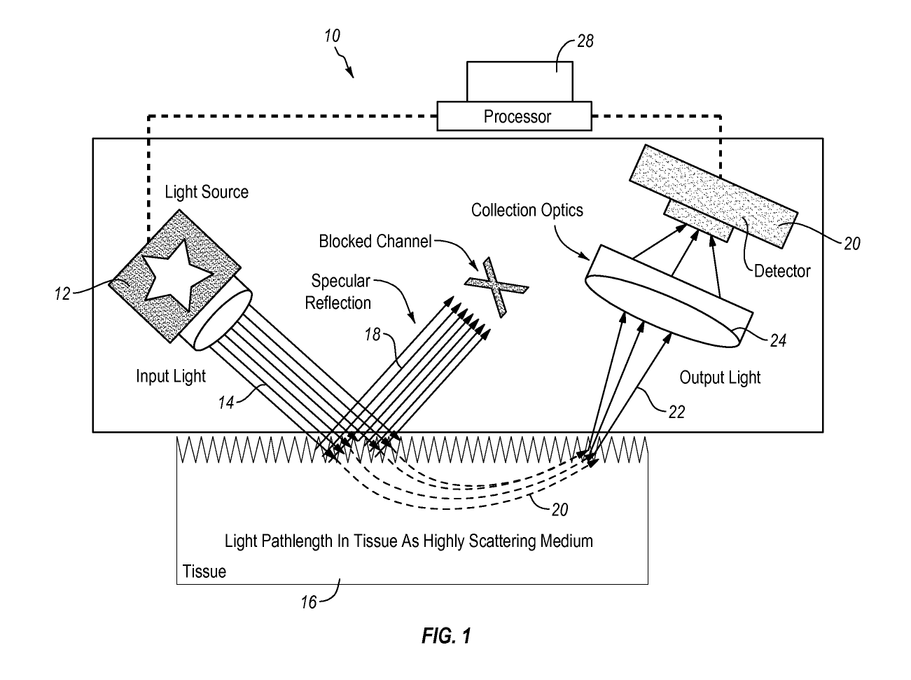

FIG. 1 is a general schematic depiction of the apparatus 10 of the present invention for measuring the transmission spectra of chemical compounds in biological tissue using combined absorption/reflection spectroscopy. The apparatus 10 contains a light source 12, which in one preferred embodiment of the invention is a light emitting diode, emitting light with a 400 nm bandwidth centered at 550 nm. Alternatively, the light source 12 may be a separate device that generates sufficient light in the spectral range of the chemical compound absorption. The device 10 further includes a light path 14 for the input light, a pathway 22 and 24 for output light, a detector 20, and a processor 28.

Incident light from the input light source 12 passes through the light path 14 and impinges on the tissue 16. The absorbed-reflected light 20 passes through the tissue 16 to where it is picked up by the pathway 22 and 24 for output light and passed on to the detector 20. The processor processes the detected signal and generates an output related to the concentration of an analyte in the tissue. As is illustrated, the device 10 configured such that detection of specular reflected light is blocked 18.

For example, in the case of carotenoids (with 400 to 520 nm absorption), the light source 12 should generate light with sufficient intensity at discrete wavelength locations or at certain spectral ranges which overlap with the absorption bands of carotenoids. Such light is readily available from light emitting diodes (LEDs).

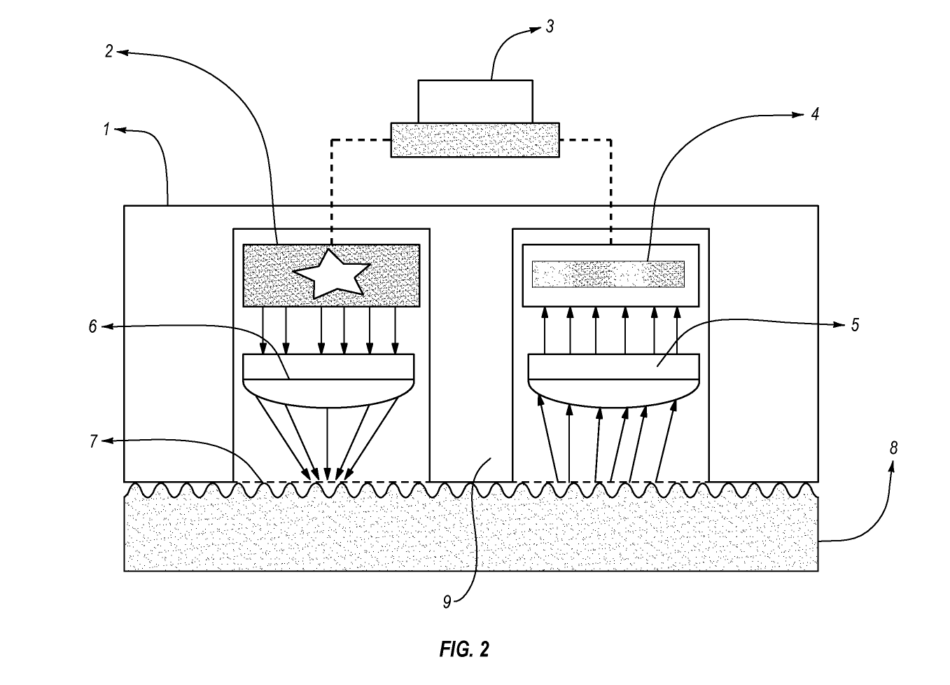

The illumination light source 12 is in optical communication with a light beam delivery 14 and collection system 22 and 24. This system can include various optical components for directing the illumination light onto the sample tissue and collecting the diffused scattered light for analysis. As illustrated in FIGS. 1 and 2, the optical components of the apparatus include the light source, the illumination module including various lenses, mirrors, optical filters or their assemblies, light delivery systems delivering illumination light from light source to the tissue and collecting the transmitted light from the tissue into the detection arrangement, a window that is placed against the tissue to be measured, a chamber or holder to hold the tissue to be measured, a light collection module including various lenses, mirrors, optical filters or their assemblies, a spectrograph, a computer processor, a monitor, and a detector, which could be either the integrated detector covering the entire spectrum (e.g. one or two-dimensional CCD/CMOS in spectrograph-based device) or arrays of single detectors (e.g. photodiodes in optical filter-based device). The interaction of these optical components with the light from the light source will be discussed in further detail below.

The detection part of the apparatus can contain a spectrometer, which serves to spectrally disperse the components of the transmitted light beam. Optical components such as diffraction gratings, prisms, dielectric filters, and different combinations of these can be used to replace the spectrometer if necessary.

The spectrally selective system is in optical communication with a light detection system, which is capable of measuring the intensity of the diffused scattered light beam as a function of wavelength in the wavelength range of interest, such as the wavelength range characteristic of carotenoid compounds in human tissue. The detection system may comprise, but is not limited to, devices such as a CCD (charge-coupled device) detector array.

The spectrally selective system and light detection system can be selected from commercial spectrometer systems, such as a low-resolution grating spectrometer employing rapid detection with a charge-coupled silicon detector array. For example, a grating spectrometer can be used that employs a dispersion grating with 300 lines/mm, and a silicon detector array with 20 μm individual pixel width. The spectrally selective system and light detection system can also be combined into an imaging system that includes spectrally selective optical elements used in association with a low light level CCD imaging array such as an intensified CCD camera.

The detected light is preferably converted by a light detection system into a signal that can be visually displayed on an output display. The light detection system may also convert the light signal into other digital or numerical formats, if desired. The resulting diffused scattered light signals are analyzed with a quantifying system, which may be calibrated by comparison with incident light. The quantifying means may be a computer, preferably one on which data acquisition software is installed that is capable of spectral manipulations and the determination of concentration values of the relevant chemical compounds. The quantifying system may also comprise a CCD image display or monitor. The quantifying system may be combined with the output display in one computer, and can calibrate the results of the chemical compound of interest obtained with other experiments such as the optical density that is proportional to actual levels.

During operation of the apparatus, a light beam is generated from the light source and is directed through an input optical fiber to the delivery system. Alternatively, the light beam can be directed to the light delivery system using mirrors. The incident light routed toward the system is expanded, filtered, and imaged with a a lens through a window onto the tissue to be measured, which will be in contact with the window. The light beam travels inside the tissue and then diffused scattered light from the tissue is collected by a lens, and is routed to a spectrally selective system such as a grating spectrograph. The spectrally dispersed light is directed to a light detection system that measures the light intensity as a function of wavelength in the relevant wavelength range. This, in turn, measures apparent absorption or optical density of the relevant chemical compounds in the tissue sample.

FIG. 2 shows the instrument details and the concept of Abs-Ref method. The whole instrument is placed inside the case (1). The illumination light from a light source (2) which could be any light source (such as sunlight, tungsten, tungsten-halogen, light emitting diode (LED) or any coherence or non-coherence light source), is routed by certain optical elements (6) on the human tissue (8), (here we consider human skin) and after travelling all outer skin layers, and scattered and absorbed by all major scatters and absorbers including antioxidant beta-carotene comes out to the surface and collected by collection optics (5), detecting by detector part (4), (which could be but is not limited to: any detector, including a spectrograph, or filter facilitated photodiodes) and analyzed by processor (3). The illumination and detection channels are separated by an opaque divider (9). If the distance between these channels increases (i.e., if the size of the divider (9) is increased), the diffuse scattering process will become dominant and as a consequence the detector channel will detect the diffuse scattering. However, if the path length (i.e., the width of the divider (9)) is too long, then the diffuse signal will be strongly attenuated and the signal to noise ration becomes small. In contrast, if the distance (i.e., the width of the divider (9)) is decreased significantly, the light from the light source will leak through the detector channel and wash out the information regarding antioxidant levels. During the measurement, both channels are in contact with tissue via transparent windows (e.g. glass, quartz or fused silica). These windows must be placed at the opening of the channels in such a way as to keep the divider and the channels extended equally.

Method of the Present Invention.

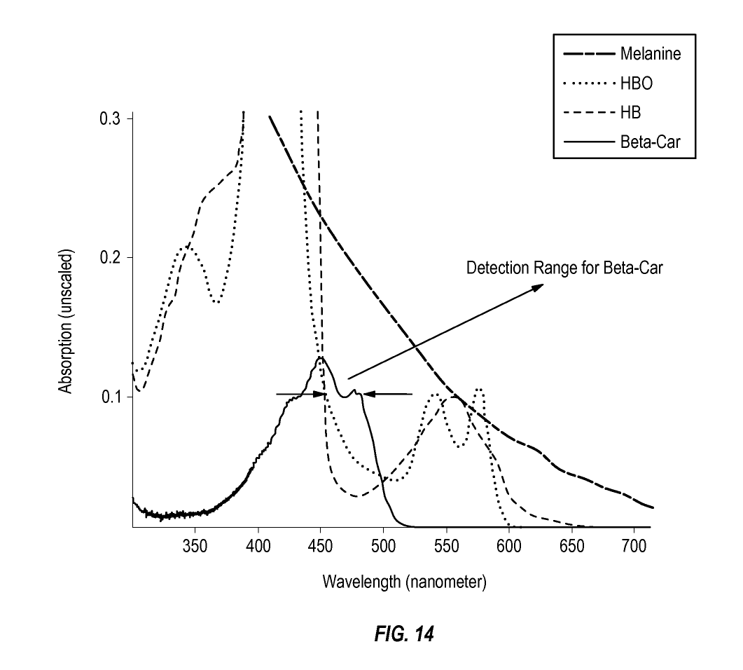

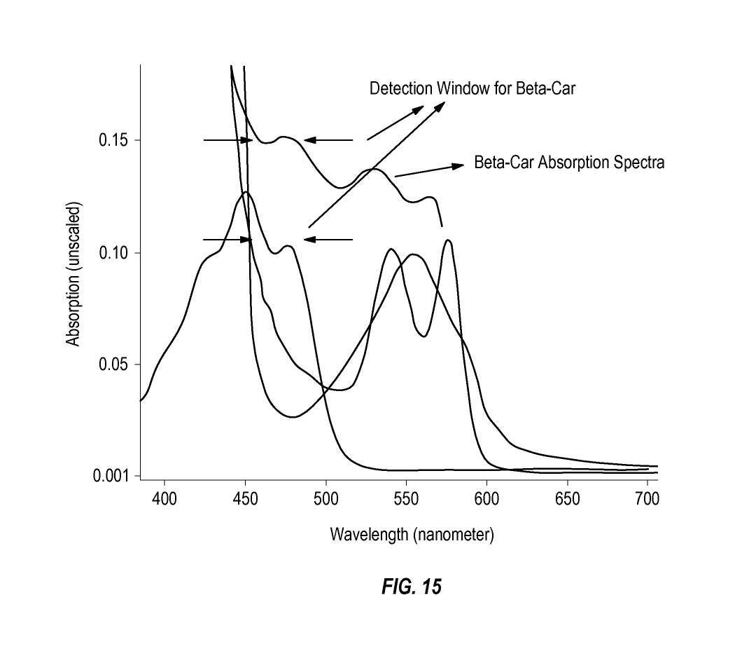

To measure the antioxidant carotenoids in human skin, the light source spectrum (I_{source}) is measured accurately and stored in the instrument's memory for future use. During the measurement, the light signal passing through the tissue and reflecting back is measured by detector channel and stored also stored in the memory (I_{Tissue}). Then the ratio of these two spectrums, in any mathematical format such as logarithmic, at a certain wavelength (which overlaps with the carotenoids absorption band, but only in the lower absorption level of blood, such as 485 nm±5 nm, as shown in FIG. 14), is proportional to the antioxidant carotenoid levels stored in the tissue, as shown in FIG. 15. This ratio, which is referred to here as “Abs-Ref result,” is obviously different for each subject who has a different level of carotenoids. FIG. 15 shows a real measurement from inside the human skin of a healthy subject, a 50 year-old male, which is overlapped on the blood and beta-carotene absorption band. This spectrum shows the two main pigments in tissue, blood and beta-carotene, and also the pertinent detection range, which as mentioned is 485 nm±5 nm. So in contrast with other reflection techniques, the present invention uses its own light source spectrum as opposed to the white standard used in other instruments.

When the Abs-Ref result is extracted from the raw data, there are many a statistical methods available to extract the level of carotenoids in human tissue. Because most of the carotenoids have absorption spectra close to beta-carotene and overlap on the beta-carotene absorption band, the Abs-Ref result can be assumed to be the outcome of most of the available carotenoids in human tissue. It should be noted here that melanin pigment absorption band varies gradually in the detection range so its optical characteristics need not be taken into account.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An instrument for measuring absorption and reflection in a biological tissue, comprising:
a housing that includes:
an incident light source;
an output light detector configured to detect scattered light from the incident light source; and
a divider separating the incident light source from the output light detector, wherein the divider is sized and configured to prevent impingement of specularly reflected light from the incident light source on the output light detector.

2. The instrument of claim 1, further comprising a processor operatively coupled to the incident light source and the output light detector.

3. The instrument of claim 2, wherein the processor is configured to compare incident light produced by the incident light source to absorbed and reflected light detected by the output light detector, and the processor is further configured to quantify at least one chemical in the biological tissue based on the comparison of the incident light produced by the incident light source to absorbed and reflected light detected by the output light detector.

4. The instrument of claim 2, wherein the processor is integral to the housing.

5. The instrument of claim 2, wherein the processor is separate from the housing.

6. The instrument of claim 1, wherein the instrument is configured for noninvasive measurement of a chemical compound in the biological tissue.

7. The instrument of claim 6, wherein the chemical compound is one or more of hemoglobin, melanin, or a carotenoid.

8. The instrument of claim 1, wherein the incident light source comprises a white light source.

9. The instrument of claim 8, wherein the incident light source produces light in a wavelength range of about 350 nm to about 750 nm.

10. The instrument of claim 1, wherein the housing further comprises one or more of lenses, optical filters, mirrors, light delivery systems configured to deliver light from the incident light source to the biological tissue, or light delivery systems configured to deliver absorbed and reflected light from the biological tissue to the output light detector.

11. The instrument of claim 1, wherein the output light detector includes one or more of a CCD chip, a CMOS chip, a photodiode array, a spectrometer, a diffraction grating, a prism, or at least one optical filter.

12. The instrument of claim 2, wherein the output light detector and the processor are configured to convert an output light signal detected by the output light detector into a readable display.

13. A method for quantifying a chemical in a biological tissue, comprising:
providing an instrument that includes a housing having:
an incident light source;
an output light detector configured to detect scattered light from the incident light source;
a divider separating the incident light source from the output light detector, wherein the divider is sized and configured to prevent impingement of specularly reflected light from the incident light source on the output light detector; and
a processor;
contacting the biological tissue with the instrument, wherein the contacting blocks the output light detector from direct detection of light produced by the incident light source;
illuminating the biological tissue with the incident light source;
detecting absorbed-reflected light that is passed through the biological tissue with the output light detector.

14. The method of claim 13, further comprising:
comparing incident light produced by the incident light source to absorbed and reflected light detected by the output light detector, and
determining a concentration of one or more chromophores in the biological tissue.

15. The method of claim 14, wherein the one or more chromophores are selected from the group consisting of hemoglobin, melanin, carotenoids, and combinations thereof.

16. The method of claim 13, further comprising:
collecting and storing a dark measurement;
collecting and storing an incident light spectrum;
contacting the instrument on the biological tissue and illuminating the biological tissue with the incident light source;
collecting and storing a measurement of absorbed and reflected light from the biological tissue;
calculating an amount of one or more chromophores in the biological tissue; and
displaying the amount of one or more chromophores in the biological tissue.
